(19) United States
(12) Patent Application Publication
(10) Pub. No.: US 2024/0299220 A1
(43) Pub. Date: Sep. 12, 2024

(54) HYGIENE ARTICLE WITH A PEEL-OFF ARRANGEMENT

(71) Applicant: RKW SE, Mannheim (DE)

(72) Inventors: Michael Scherer, Flintsbach am Inn (DE); Leonhard MAIER, Babensham (DE); Manuel PAUKNER, Schonstett (DE)

(73) Assignee: RKW SE, Mannheim (DE)

(21) Appl. No.: 18/574,813

(22) PCT Filed: May 5, 2022
(86) PCT No.: PCT/EP2022/062090

§ 371 Date: Dec. 28, 2023

(30) Foreign Application Priority Data
Jun. 29, 2021 (DE) 10 2021 116 730.2

(51) Int. Cl. A61F 13/56

(52) U.S. Cl. CPC A61F 13/5611

(57) ABSTRACT

A hygiene article having an arrangement for peeling off a layer from an adhesive layer. The arrangement comprises a carrier layer and a separation layer. The carrier layer has at least one β-nucleated, stretched, polyolefinic layer having a microporous structure for generating an opacity.

### CROSS-REFERENCE TO RELATED APPLICATIONS

This application is a 371 National Phase of PCT/EP2022/062090, filed May 5, 2022, which claims priority from German Patent Application No. 10 2021 116 730.2, filed Jun. 29, 2021, both of which are incorporated herein by reference as if fully set forth.

### TECHNICAL FIELD

The invention relates to a hygiene article having an arrangement for peeling off a layer from an adhesive layer, the arrangement comprising a carrier layer and a separation layer.

### BACKGROUND

The term hygiene articles covers the use of articles that women use during menstruation, during postpartum flow, or on an ongoing basis to protect lingerie from blood, cervical mucus, vaginal secretions, and other natural excretions. Such hygiene products include the product categories sanitary towels, panty liners, tampons, menstrual sponges and period underwear.

The invention relates to products that are attached to the underwear during use. Typical examples include sanitary napkins, panty liners, and incontinence pads. These absorbent articles typically include a topsheet, a backsheet, and optionally an absorbent core disposed between the topsheet and the backsheet, and a fastening adhesive applied to the garment-facing surface of the backsheet.

Such hygiene articles are worn, for example, in the gusset of an undergarment. The carrier layer of the sanitary article preferably has an adhesive to position it in place in the undergarment. Some models of the have wing flaps that can be folded around the gusset of the undergarment to provide additional stability.

Such a prior art hygiene product is described, for example, in U.S. Pat. No. 4,347,092. The product in question is a panty liner. For its production, an absorbent pad and a liquid-permeable cover are applied to one layer.

In order to keep the adhesive ready for use until such a pantyliner is used, it is covered with an arrangement for peeling. Such an arrangement for peeling is often in the form of a separation paper which is provided with a coating to enable peeling off easily and without damaging the hygiene article.

EP 1 138 294 B2 describes a coated paper separation strip that can be applied to the adhesive side of a hygiene article to protect the adhesive before its use. The coating, such as silicone, reduces the adhesion of the coated side of the separation strip to the adhesive. Conventional separation strips are often based on a paper material that adheres to the adhesive with sufficient toughness due to the coating, but which can be readily removed when the sanitary article is in use. To ensure sufficient stiffness, such paper strips have a high basis weight, usually of more than 35 g/m2.

Such arrangements for peeling off a layer from an adhesive layer often have a separation layer, also known as a release coating, based on a polysiloxane, for example.

In addition, it is usually desirable to print such protective covers. This is done for informative purposes or for aesthetic or promotional reasons.

DE 10 2018 101 331 A1 discloses a separation film comprising a carrier layer, a printed image and a release coating. The printed image is at least partially disposed between the carrier layer and the release coating and comprises at least one ink cured by UV radiation. The release coating comprises a polysiloxane cured by UV radiation.

Conventional arrangements for peeling can cause rustling noises during peeling. In addition, they are often problematic in terms of environmental protection, as they are made from combinations of materials that are difficult to recycle. Coloring based on titanium dioxide pigments is often used to provide a white printing base. For health reasons, the aim is to avoid the use of these inorganic pigments in hygiene products.

One important goal is to minimize the environmental impact of disposable hygiene articles. To conserve raw material resources, the lightest and thinnest possible layers are used. The use of single-variety materials is also pursued to facilitate recycling.

### SUMMARY

The object of the invention is to provide a hygiene article that meets the highest health and ecological requirements. The article is to have an arrangement which, on the one hand, reliably covers an adhesive layer but, on the other hand, can be removed easily and quietly during use. In addition, the arrangement should be printable to create a high-quality design.

This object is solved according to the invention by a hygiene article with an arrangement for peeling, a method and a use according to the subordinate main claims. Preferred variants are to be taken from the subclaims and the description.

According to the invention, the arrangement for peeling off a layer from an adhesive layer comprises a carrier layer comprising at least one B-nucleated, stretched, polyolefinic layer having a microporous structure for generating an opacity.

In a particularly advantageous variant of the invention, the layer has a polypropylene component whose polypropylene content is preferably more than 90%by weight. Propylene homopolymers and/or propylene block polymers have proved particularly suitable for this purpose.

Ideally, the carrier layer is formed from almost 100% polypropylene. This grade purity facilitates recycling.

The flow behavior of polypropylene is described with the aid of the melt index MI (Melt Index), usually at a temperature of 230° C. and a load of 2.16 or 5 kg. A higher melt index correlates with a lower average molecular weight of the polymer. At the same time, the higher the melt index of a polymer, the lower the melt viscosity. On the other hand, polymers with a high molecular weight, i.e. a low melt flow index, are advantageous in terms of mechanical stability, especially tensile strength or toughness.

In an advantageous embodiment of the invention, the polypropylene has a density of 0.9 g/cm³ and a melting range of 155 to 165° C. The heterophasic polymer preferably has a melt flow index of less than 15 g/10 min, preferably less than 10 g/10 min, in particular less than 5 g/10 min, in each case at 230° C. and 2.16 kg.

The specially selected polypropylene, preferably with a Charpy impact according to DIN EN ISO179-1 of more than 30 KJ/m², preferably more than 35 KJ/m2, in particular more than 40 KJ/m2, for example a Repsol Isplen PB120G1F, is extruded into a thin layer. The carrier layer produced has a high stiffness, which is a prerequisite for easy peeling of the arrangement with a good “finger lift”.

According to the invention, nucleating agents are used in at least one layer of the carrier layer. An adjustment of spherulite sizes of the selected polymers with a stretching leads to an opacity that ensures an appealing print design, while at the same time ensuring particularly favorable mechanical properties of the carrier layer.

Particularly in the case of isotactic polypropylene, it is possible to specifically nucleate the α-crystal modification or the β-crystal modification using suitable nucleating agents. α- and β-nucleated polypropylene differ greatly in mechanical and optical properties. In addition, a few B-nucleating agents also succeed in improving the optical properties such that an opaque polypropylene layer can be obtained.

In an advantageous variant of the invention, at least one layer of the carrier layer has a proportion of β-spherulite nucleating agent. Here, the proportion of β-spherulite nucleating agent is more than 1 ppm, preferably more than 2 ppm, in particular more than 3 ppm. It has been shown that the use of B-spherulite nucleating agent can be carried out very sparingly and that the proportion is less than 1000 ppm, preferably less than 500 ppm, in particular less than 50 ppm.

As β-spherulite nucleating agent, a substance called Cinquasia Gold, a substituted quinacridone, is preferably suitable due to its excellent dispersibility in polymer blends. Here, a few ppm of Cinquasia Gold, preferably 10 ppm of Cinquasia Gold, added to the polymer blend in the extruder is sufficient to produce an extremely fine crystalline polypropylene layer. Subsequent stretching of the layer results in the layer exhibiting strong opacity by forming a microporous structure. In principle, all known quinacridone pigments can be used as B-spherulite nucleating agents.

The carrier layer is either stretched in machine direction only (MD), or both machine and cross direction (CD). This creates a microporous structure in the layer, which leads to the desired opacity.

The carrier layer can, for example, be stretched with a stretch ratio of 1:1.5 in the machine direction. It is also possible to subject the film web to additional transverse stretching (CD).

In a particularly advantageous variant of the invention, sufficient opacity is produced without the use of an inorganic filler in the carrier layer, preferably without incorporating a white pigment. According to the invention, titanium dioxide in particular can be dispensed with. This is advantageous from both health and ecological aspects.

In an extremely advantageous embodiment of the invention, the arrangement for peeling has no titanium dioxide, preferably no white pigment, and particularly preferably no inorganic filler for generating opacity. This embodiment meets the requirements of environmental protection and health protection to a particular degree.

Opacity is the opposite of transparency. It is a measure of the opacity or opacity in percent. In particular, the opacity of a completely opaque layer is 100% and a completely or totally transparent layer has an opacity of 0%.

In particular, opaque plastic layers are just not transparent in the wavelength range of visible light. Advantageously, the carrier layer has an opacity according to DIN 53416 of more than 45%, preferably more than 60%, in particular more than 75%.

The pronounced opacity based on a polypropylene-based carrier layer enables printing on a printing layer with innovative designs. The arrangement for peeling according to the invention is characterized by the fact that it can be produced completely free of inorganic fillers and commonly used white pigments.

Here, the arrangement for peeling proves to be particularly pleasant due to the especially soft and at the same time stiff carrier layer made of special polypropylene during the so-called “finger lift”. Conventional paper separation strips do not provide such an effect.

The arrangement according to the invention has a particularly low noise emission when being pulled off the hygiene article and maintains discretion to a special degree.

In an advantageous variant of the invention, the arrangement for peeling is particularly thin and has a low basis weight. In this case, the carrier layer comprises at least two layers, the carrier layer having a basis weight of less than 35 g/m², preferably less than 30 g/m², in particular less than 25 g/m², and/or more than 10 g/m², preferably more than 14 g/m², in particular more than 18 g/m². As a result, the arrangement for peeling has a significantly lower weight per unit area compared with known separation layers, in particular paper separation strips. This is particularly advantageous for reducing the amount of material used with regard to environmental aspects.

The arrangement for peeling has a separation layer to support the peeling process. It has proved advantageous if at least one layer of the separation sheet has a water vapor permeability of less than 100 g/m², in 24 h according to ASTM D6701-01. This makes the layer virtually airtight.

For this purpose, the separation layer comprises a polysiloxane, preferably a polydimethylsiloxane. For the purposes of the invention, the term polysiloxane refers to compounds whose polymer chains are composed alternately of silicon and oxygen atoms. A polysiloxane is based on n recurring siloxane units (—[Si(R2)—O]—)n, each of which is independently disubstituted with two organic radicals R, where R preferably represents R1 or OR1 in each case and R1 represents an alkyl radical or an aryl radical in each case.

Preferably, the polysiloxane applied for the separation layer is based on a recurring di-alkylsiloxane unit or on a recurring alkylaryl-siloxane unit. Depending on how many Si—O bonds an individual siloxane unit has, in each case based on a tetravalent silicon atom, these units can be differentiated into terminal monofunctional siloxanes (M) with one Si—O bond, difunctional siloxanes (D) with two Si—O bonds, trifunctional siloxanes (T) with three Si—O bonds and tetrafunctional siloxa-ne (Q) with four Si—O bonds.

Preferably, the polysiloxane used has a crosslinked ring-or chain-like, especially preferably a crosslinked chain-like structure, which is linked by (D)-, (T)-, and/or (Q)-units to form a two-or three-dimensional network.

The basis weight of the separation layer in the form of a coating of the carrier layer, preferably of polydimethylsiloxane, is preferably less than 0.6 g/m², in particular less than 0.5 g/m², preferably less than 0.4 g/m². Compared to previously known paper separation strips, the separation layer is formed with an extremely low basis weight. The arrangement according to the invention is particularly advantageous from the point of view of environmental protection, since the weight per unit area is reduced by more than half compared with known paper separation strips.

It has proved advantageous that at least one layer of the carrier layer, preferably the layer of the separation layer facing the carrier layer, is not microporous. As a result, the separation layer adheres much better to the carrier layer and performs its object with a significantly reduced basis weight compared to known paper separation strips.

In one embodiment of the invention, the arrangement for peeling has a partially embossed structure, whereby an improvement in the separation effect from the adhesive layer can be achieved compared to conventional separation films.

In an advantageous embodiment, the carrier layer has a single-sided coating based on a polysiloxane, wherein the carrier layer also comprises an embossed structure.

The embossing structure is preferably based on a recurring and/or regularly arranged pattern. The embossing structure may be a continuous embossing structure, such as a continuous groove structure, or several preferably recurring individual embossing structures.

Preferably, each embossing structure is based on a plurality of recurring individual embossing structures. These respective individual embossing structures can preferably be based on a groove structure, which have more or less pronounced embossing elevations, by which the embossing height of the embossing structure is defined. According to the respective geometry of the webs of a preferably recurring individual embossing structure, a plurality of respectively different, preferably recurring, individual embossing structures can result in the top view, such as for example preferably serpentine, serrated, hexagonal, diamond-shaped, rhombic, parallelogram-shaped, honeycomb-shaped, circular, dot-shaped, star-shaped, linen-shaped, reticulated, polygonal, preferably triangular, square, particularly preferably triangular, rectangular, particularly preferably triangular, rectangular, particularly preferably rectangular, quadrangular, particularly preferably rectangular and square, pentagonal, hexagonal, heptagonal and octagonal, wire-shaped, elliptical, oval and grid-shaped patterns, whereby at least two patterns can also overlap.

The embossing webs can each preferably have a length of up to one centimeter, and a length of 0.001 mm to 10 mm is particularly preferred. The proportion of the webs to the total length of the grooved structure is preferably less than 75%, particularly preferably from 5% to 60% and especially preferably from 10% to 50%. Preferably, the webs are arranged at regular or alternating, recurring intervals. The webs of the individual embossing structures can also have a curvature, i.e. a convex and/or or concave structure.

Preferably, a uniform embossing height of the layer is produced. At least the side of the carrier layer facing the separation layer has an embossed structure, the average embossing height being more than 5 μm, preferably more than 10 μm, in particular more than 15 mμ.

Alternatively, pressure embossing of the arrangement for peeling can cause recrystallization to start in the β-spherulite crystallite structure of the extruded polypropylene. As a result, the layer of the carrier layer appears transparent again in a defined manner at the embossing points. In this resource-saving way, preferably promotionally effective motifs can be produced on the arrangement for peeling off the hygiene article. Pressure embossing is carried out at significantly higher pressures than embossing an embossing structure.

In one variant of the invention, the arrangement is irrevocably marked using heat and/or pressure. This is one way to protect articles from product piracy. Unlike printing, which can be removed with a suitable solvent, it is not possible to restore the pore structure of an arrangement marked in this way, which has been specifically destroyed by pressure and heat.

Laser radiation can be used for such an action to change the pore structure. Alternatively or complementarily, the pore structure can be changed by the action of ultrasound.

In a particularly preferred variant of the invention, the arrangement for peeling has a print. The print is preferably in the form of a print motif and/or a printing layer. In the field of hygiene articles, the term print motif refers to the manufacturer-identifying and thematic design part of a print. Unlike a printing layer, an overprint is only a partial application.

Preferably, a print is applied using a flexographic printing process. The printing inks to be used for printing are preferably printing inks with a low viscosity and are thus almost water-thin. In principle, water-based, UV-curable and solvent-based ink systems are suitable. These printing inks can be specially adapted to the respective printing process and therefore individually adapted to the respective printing press. The printing inks known to the skilled person can be dried or cured in particular by heat and/or UV light.

In an alternative embodiment of the invention, the print is applied to the carrier layer of the arrangement for peeling using a digital printing process. In digital printing, the printed image is transmitted to a printing machine in the form of digital data. The great advantage of digital printing is to dynamically generate an image dot addressing within the print format for each individual printing process. This means that each printing layer can also be individualized and/or even personalized.

In an advantageous embodiment of the arrangement for peeling according to the invention, the print can be arranged on the side of the carrier layer facing away from the separation layer and thus applied to the directly visible surface of the arrangement. Alternatively, the printing layer can be arranged between the carrier layer and the separation layer and can partially show through the transparent areas of the print embossing.

Separation papers known in the prior art for peeling off a layer from an adhesive layer of hygiene articles usually have basis weights of more than 45 g/m2 and are coated with silicone, the basis weight of which is more than 0.8 g/m2. The arrangement for peeling according to the invention is characterized by a recyclable carrier layer which, due to its low weight per unit area, requires significantly less material and at the same time exhibits good peel-off behavior from the adhesive layer, the applied silicone layer having a weight per unit area which is reduced by more than half. The arrangement for peeling according to the invention makes a major contribution to making hygiene products more sustainable.

The arrangement for peeling is preferably produced by a process in which a specific composition is first extruded to form a carrier layer, preferably the carrier layer is a coextruded polypropylene carrier layer. The extrusion is preferably carried out as a blow extrusion and is cooled for further processing. The carrier layer is then stretched in the machine direction. According to the invention, the application of the separation layer and, if necessary, the printing layer is carried out in an inline process. In the inline process, the film web does not have to be rolled up and unrolled again before the separation layer and/or the printing layer are applied.

The arrangement according to the invention for peeling off a layer from an adhesive layer for hygiene articles is used in particular as a release liner for disposable hygiene articles, for example for sanitary towels and panty liners.

1. A hygiene article, comprising:
an arrangement for peeling off a layer from an adhesive layer, having
a carrier layer and
a separation layer,
and the carrier layer comprises at least one B-nucleated, stretched, polyolefinic layer with a microporous structure for generating an opacity.

2. The hygiene article as claimed in claim 1, wherein the layer comprises a polypropylene component, wherein a proportion of polypropylene is more than 90% by weight.

3. The hygiene article as claimed in claim 1 2, wherein the layer comprises a proportion of b-spherulite nucleating agent, wherein a proportion of b-spherulite nucleating agent is more than 1 ppm and less than 1000 ppm.

4. The hygiene article as claimed in claim 1, wherein the carrier layer comprises at least one layer having a water vapor permeability of less than 100 g/m² in 24 h according to ASTM D6701-01.

5. The hygiene articles as claimed in claim 1, wherein the carrier layer has a basis weight of less than 35 g/m²and more than 10 g/m².

6. The hygiene article as claimed in claim 1, wherein the separation the and a basis weight of the separation layer is less than 0.6 g/m².

7. The hygiene article as claimed in claim 1, wherein the layer has no titanium dioxide, no white pigment, and no inorganic filler.

8. The hygiene article as claimed in claim 1, wherein the arrangement has no titanium dioxide, preferably no white pigment, no inorganic filler for producing opacity.

9. The hygiene article as claimed in claim 1, wherein the carrier layer has an opacity according to DIN 53416 of more than 45%.

10. The hygiene article as claimed in claim 1, wherein the arrangement comprises a print.

11. The hygiene article as claimed in claim 1, wherein at least a side of the carrier layer which is facing the separation layer has an embossed structure with an average embossing height of more than 5 mm.

12. The hygiene article as claimed in claim 1, wherein the carrier layer has an area with a change in a pore structure achieved by at least one of laser radiation or ultrasound for marking.

13. A method of manufacturing an arrangement comprising the following steps:
preparing a composition;
extruding the composition as a carrier layer;
stretching the carrier layer; and
coating the carrier layer with a separation layer.

14. The hygiene article of claim 1, wherein the arrangement comprises a release liner for peeling off an adhesive layer.
